(19) United States
(12) Patent Application Publication
(10) Pub. No.: US 2013/0072838 A1
(43) Pub. Date: Mar. 21, 2013

(54) THERAPY CAST

(71) Applicants: Gwenyth Fischer, Minneapolis, MN (US); Dawn Bardot, Saint Paul, MN (US); Michael Dahl, Eden Prairie, MN (US); Kiyoyuki Miyasaka, Tokyo (JP); Larry Nilsson, Burnsville, MN (US)

(72) Inventors: Gwenyth Fischer, Minneapolis, MN (US); Dawn Bardot, Saint Paul, MN (US); Michael Dahl, Eden Prairie, MN (US); Kiyoyuki Miyasaka, Tokyo (JP); Larry Nilsson, Burnsville, MN (US)

(73) Assignee: Gradcast LLC, Burnsville, MN (US)

(21) Appl. No.: 13/529,930

(22) Filed: Jun. 21, 2012

Related U.S. Application Data
(60) Provisional application No. 61/500,048, filed on Jun. 22, 2011.

(51) Int. Cl. A61F 13/04

(52) U.S. Cl. CPC A61F 13/04
USPC 602/2

(57) ABSTRACT

A therapy cast can provide fixation, compression, or thermal therapy to a patient. The therapy cast can include a support structure configured to couple to a lower leg of a person and configured to at least partially restrict movement of the lower leg. A compression member can be configured to exert a compressive force to a first portion of the lower leg. The compressive force can include a gradient relative to a spatial position of the lower leg. A thermal member can provide thermal therapy to a second portion of the lower leg.

### CLAIM OF PRIORITY

This patent application claims the benefit of priority, under 35 U.S.C. Section 119(e), to Gwenyth Fischer et al., U.S. Provisional Patent Application Ser. No. 61/500,048, entitled “Therapy Cast,” filed on Jun. 22, 2011 (Attorney Docket No. 3687.001PRV), which is hereby incorporated by reference herein in its entirety.

### BACKGROUND

Fixation of limbs can be used to prevent further damage after an injury or surgery. Fixation can be provided by a device, cast, or in trauma situations, an intramedullary nail or rod. Fixation devices can be configured to fit a number of anatomies, including neck, arm, writs, leg, or foot.

### OVERVIEW

This overview is intended to provide an overview of subject matter of the present patent application. It is not intended to provide an exclusive or exhaustive explanation of the invention. The detailed description is included to provide further information about the present application.

The present inventors have recognized, among other things, that fixation, compression, and thermal therapy can promote healing of an injury to an appendage. Application of each treatment separately or in combination can be accomplished with a single device.

In an example, a device can comprising a support structure configured to couple to a lower leg of a person and configured to at least partially restrict movement of the lower leg, a compression member configured to exert a compressive force to a first portion of the lower leg, the compressive force having a gradient relative to a spatial position of the lower leg, and a thermal member configured to provide thermal therapy to a second portion of the lower leg.

In an example, the wearable support structure can include a boot back, a boot, and a cover.

In an example, the boot back can include a support beam configured to provide stability.

In an example, the cover can include a hinge configured to couple the cover to the boot.

In an example, the boot back can include a hinge configured couple a first portion of the boot back to a second portion of the boot back.

In an example, wherein the compression member can include at least one bladder.

In an example, a bladder can include a push-button release configured to vent the bladder to atmospheric pressure.

In an example, the at least one bladder can include at least one relief valve.

In an example, the relief valves can be connected in parallel.

In an example, the relief valves can be connected in series.

In an example, the gradient can include a low pressure region about an upper area of the lower leg.

In an example, the gradient can include a high pressure region around a toe area of the lower leg.

In an example, the thermal member can include a thermal contact area configurable by the compressive force.

In an example, the compression member can be configured to adjust the thermal contact area between the thermal member and the lower leg.

In an example, the thermal member can be configured to provide temperature therapy using a chemical reaction.

In an example, the thermal member can be configured to provide temperature therapy using at least one of electric cooling, resistive heating, a Peltier method, radiation, friction or vibration heating, and ultrasound.

In an example, a therapy method for a lower leg can comprise stabilizing a lower leg of a person using a support structure configured to couple to the lower leg to at least partially restrict movement of the lower leg, compressing a first portion of the lower leg using a compression member configured to exert a compressive force to at least the first portion of the lower leg, the compressive force having a gradient relative to a spatial position of the lower leg and thermally treating a second portion of the lower leg using a thermal member configured to provide thermal therapy.

In an example, the method can comprise using the compression member to adjust a thermal contact area between the thermal member and the lower leg.

In an example, the method can comprise circulating a thermal therapy fluid through the thermal member using a pump.

In an example, a device can comprise a support structure configured to couple to a lower leg of a person to at least partially restrict movement of the lower leg. The support structure can include a boot back formed of a rigid material, a boot configured to couple to the boot back, and a cover configured to couple to the boot. One or more bladders configured to exert a compressive force to the lower leg, each bladder including an inlet relief valve configured in series or parallel to provide a compression gradient relative to a spatial position on the lower leg and a thermal member configured to provide thermal therapy to at least a portion of the lower leg.

In an example, the device can comprise a pump configured to pressurize the one or more bladders.

In an example, the device can comprise at least one fastener configured to couple the support structure to the lower leg.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed in the present document.

FIG. 1 illustrates an example of a therapy cast according to the present disclosure.

FIG. 2 illustrates an exploded view of a therapy cast according to the present disclosure.

FIGS. 3, 4A, and 4B illustrate examples of a boot back according to the present disclosure.

FIG. 5 illustrates an example of a boot according to the present disclosure.

FIGS. 6A, 6B, and 6C illustrate example of configurations of a plurality of compression members according to the present disclosure.

FIG. 7 illustrates an example of a therapy cast according to the present disclosure.

FIG. 8 illustrates an example of a therapy sock according to the present disclosure.

FIG. 9 illustrates a cross sectional view of an example therapy cast according to the present disclosure.

### DETAILED DESCRIPTION

In the following description, reference is made to the accompanying drawings that form a part hereof, and in which is shown by way of illustration specific embodiments which can be practiced. These embodiments are described in sufficient detail to enable those skilled in the art to practice the invention, and it is to be understood that other embodiments can be utilized and that structural, logical and electrical changes can be made without departing from the scope of the present invention. The following description of example embodiments is, therefore, not to be taken in a limited sense.

The present subject matter includes a removable therapy cast that incorporates fixation, compression, and temperature therapy. Various examples of the present subject matter can be configured to provide fixation, compression, and temperature therapy to a number of injury regions, including a knee, a shoulder, an elbow, a wrist, and other anatomical areas. The cast can be wearable, such that it can be easily put on or around a patient or easily removed from a patient.

Clinically appropriate compression or cooling, in addition to restriction of mobility, can be helpful to meet a clinician's therapy recommendations. The patient population can include surgery or trauma patients who have any estimated recovery time of about 4-12 weeks. These patients can benefit from fixation to immobilize an injured area, gradient compression to prevent accumulation of vascular fluid, or cooling to reduce swelling and provide comfort. For example, a lower leg therapy cast can be suited for use by patients recovering from, but not limited to, mid-foot fusions, soft tissue surgeries, fractures of the calcaneus, lateral malleolus, posterior malleolus or medial malleolus.

An example presented herein combines various functions in a mobile, cost-effective design. In one example, fixation can be provided by a rigid acrylonitrile butadiene styrene (ABS) 30% glass fiber-filled plastic boot back or boot back. Gradient compression, in one example, can be provided by a system of relief valves on one or more plastic air bladders. Temperature or thermal therapy, in one example, can be provided by a custom thermal member, an operable thermal compartment, and a cotton-insulation sleeve.

One example allows minimal deflections of the therapy cast (e.g., device) to ensure fixation of the injury. An example can be configured to provide a particular target skin temperature. Fixation of the lower leg after a surgical procedure or traumatic injury can allow the affected area to heal properly without interruption. Fixation can provide stability and protection to an otherwise susceptible region. Without clinically appropriate fixation, a patient can experience improper healing or increase their chances of further injury.

Construction of Therapy Cast

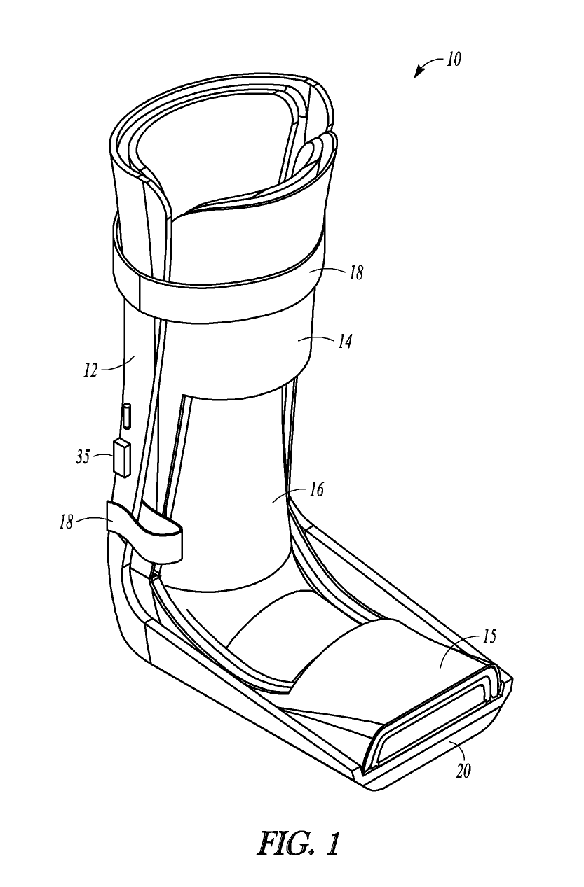

An example of the present subject matter is configured to help aid the healing process of an injured foot, ankle, or lower leg of a patient. FIG. 1 illustrates an example of a therapy cast 10 for a lower leg according to the present disclosure. Fixation can be sufficient to maintain the foot at, for example, 90° to the ankle in order to allow the foot to heal properly. If the foot is not kept in a fixed position for the extent of the healing process, the ankle or foot may not heal properly.

The ability to remove the device 10 from the lower leg can add to patient comfort by allowing the patient to remove his or her foot for cleaning purposes, while also allowing air flow around the leg. In an example, the weight of the device 10 is light enough to permit the patient to be mobile, such as walking or moving around with crutches if they are not allowed to bear weight on their foot. If the device 10 were too heavy, the patient's ability to move could be diminished, and the patient could get discouraged from wearing the device.

In an example, the present subject matter includes a boot back 12. The boot back 12 can be fabricated of various materials such as a polymer, a plastic, a composite, or a metal. In an example, the boot back is formed of a rigid material, such as a plastic of at least about 30% ABS. The boot back 12 can be configured to retain the patient's lower leg in a position deemed clinically healing.

In an example, the boot back 12 can be constructed in one or more modular units so that desired parts can be used as deemed appropriate by a clinician or patient. A modular configuration can allow for a range of anatomy to be fitted or pathology to be treated with the device. For example, amputates, or those with deformities like clubfoot can make use of the device configured to accommodate them.

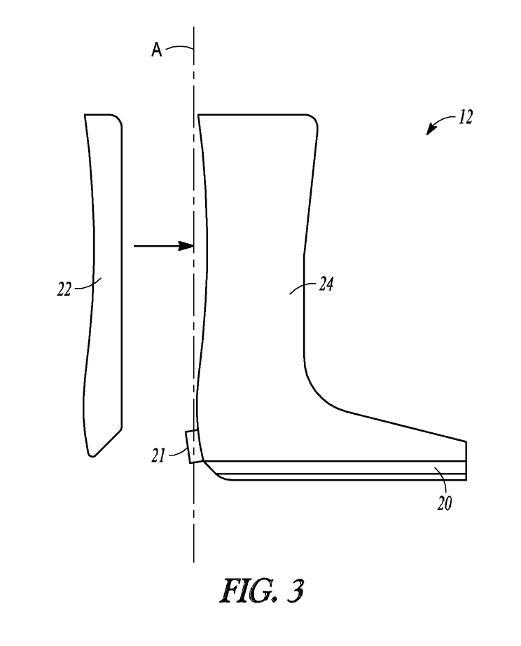

In one example, the device 10 can be placed on the foot using a variety of means. In various examples, entry is from the front or the back of the device, with the appropriate panel then attached after. As illustrated in FIG. 3, the boot back 12 can also be constructed of a first portion 22 and a second portion 24. The first and second portions 22 and 24 can couple to form boot back 12. In an example, the first or second portions 22 and 24 can include a hinge 21 that configured to permit the first portion 22 to articulate about an axis A. For example, the first portion 22 can articulate so as to open perpendicular or parallel with the second portion 24. In an example, the first portion 22 and the second portion 24 can snap fit or press fit, so as to form boot back 12. Benefits of such examples include ease of insertion of a limb into the device. Modular pieces and panels, such as a footbed or sole, as described herein, can be positioned after the boot back 12 is around the lower limb.

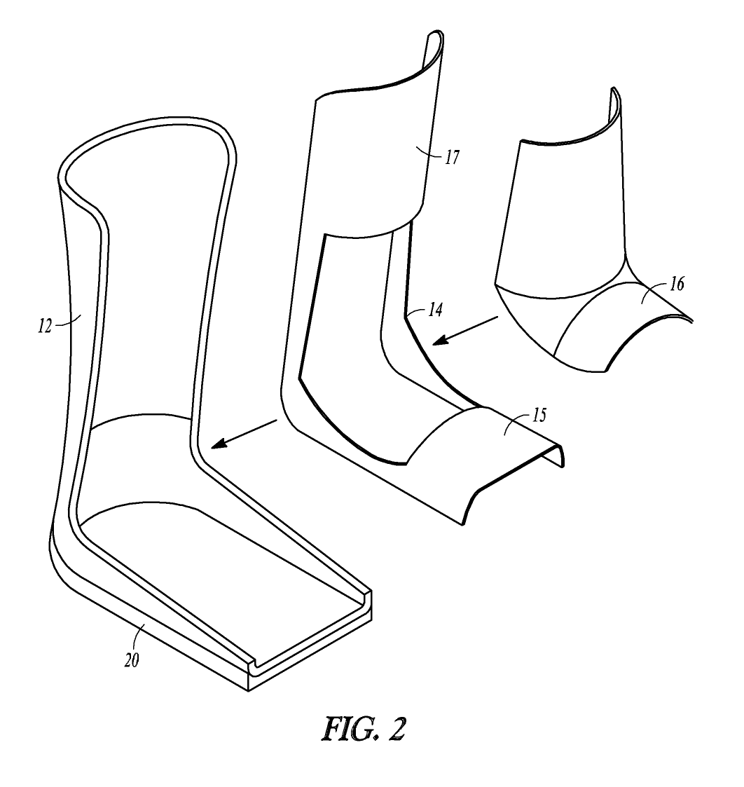

The device 10 can include a boot 14 configured to couple to the boot back 12, as illustrated in FIG. 2. The boot 14 can snap fit or press fit to the boot back 12. In an example, the boot 14 can rest on or within the booty back 12, such that it can be secured by one or more fastening devices 18 of FIG. 1, as described herein. The boot back 12 can be formed of a rigid material as described herein. As illustrated in FIG. 1 and FIG. 5, a forefoot of the boot 14 can include a component 15 that encompasses a particular anatomy such as a toe. The component 15 can apply fixation, pressure, or thermal treatment. The forefoot component 15 can facilitate application of gradient pressure in the device in small anatomy such as the toes. It can also enable fixation of the toes to assist healing from injury or surgery. As illustrated in FIG. 2, the boot 14 can include an upper component 17 that at least partially encompasses a particular anatomy such as a shin, ankle, or calf. However, as illustrated in FIG. 5, upper component 17 is optional. The upper component 17 can aid in fixation, pressure, or thermal treatment, as described herein.

The boot 14 can be formed so as to restrict movement or flexation of a patient's lower leg, such as a joint, ligament, tendon, muscle, or the like. The boot 14 can help position the lower leg as directed by a clinician. For example, the boot 14 can be at an angle less than 90°, as illustrated in FIG. 5, to help prevent a patient flexing their foot or ankle in an undesirable manner for healing. In various examples, the device 10 or boot 14 can have multiple selectable angles, including a fixed 90 degree position. Such a configuration can allow the device 10 to be set at various positions to allow patients with various pathologies, anatomies, or injuries to benefit from the device.

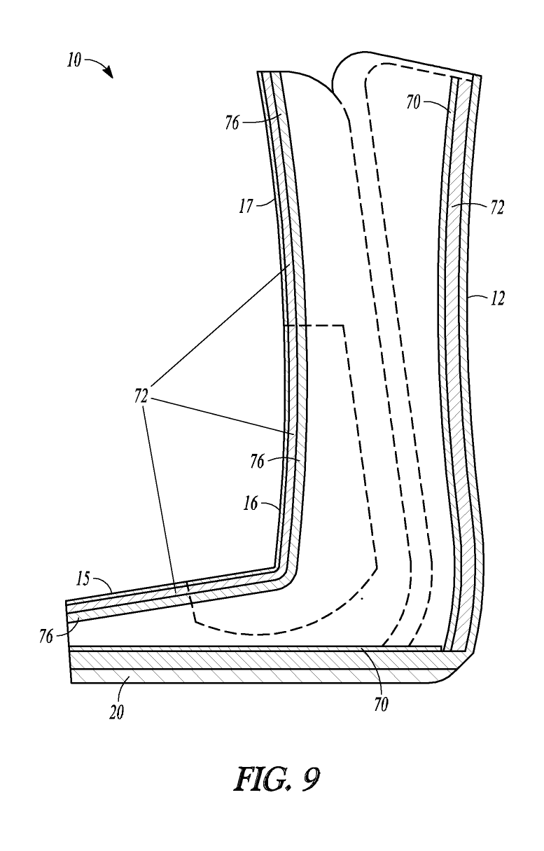

As illustrated in FIG. 2, a cover 16 can be configured to couple with the boot 14. The cover 16 can be shaped or positioned to aid in receiving a thermal member, as described herein. The cover 16 can be coupled to the boot 14 by a snap fit, a press fit, a hinge, or other coupling mechanism. The cover 16 can be formed of a rigid material, as described herein. In an example, the cover 16 can be configured to contain a thermal member, as described in relation to FIG. 9.

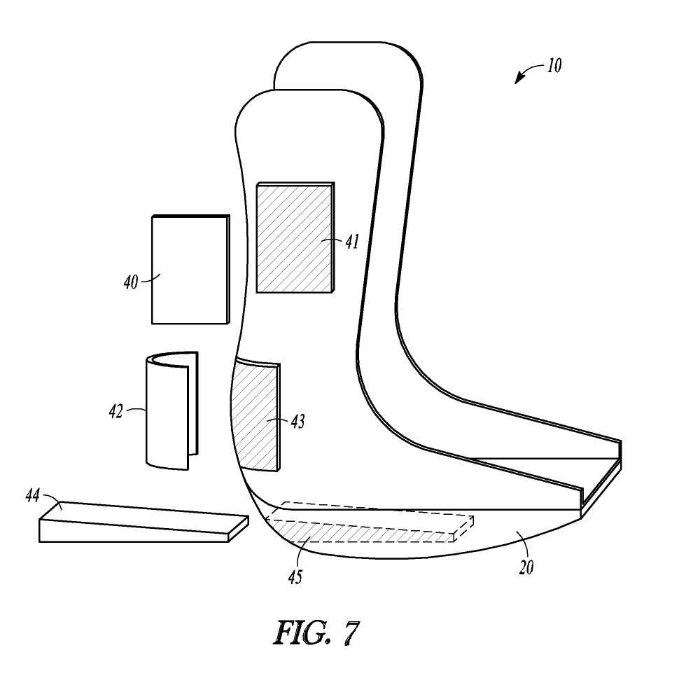

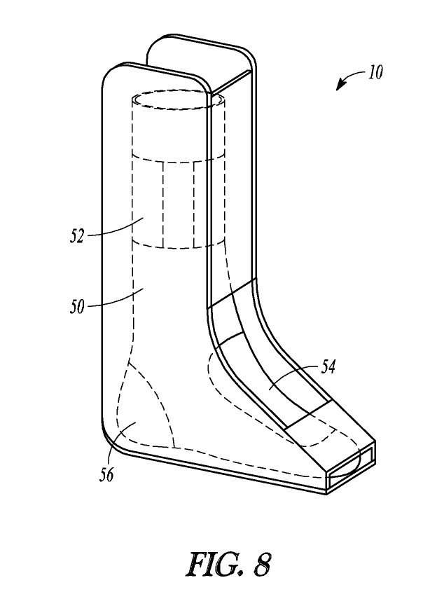

As illustrated in FIG. 1, the sole 20 of the device 10 can include a cross section of any geometry, such as flat, rounded, polygonal, or the like. For example, as illustrated in FIG. 1, the sole 20 includes a rounded cross section. In an example, the sole 20 can include a floor contact surface of any geometry. For example, the floor contact surface can be flat or rounded. A rounded floor contact surface can provide a rolling pivot configured to reduce stress on the device or increase mobility of the patient. The sole 20 can include a shock absorber, such as a foam, spring, air cushioning, or gel. The sole 20 can include a panel or compartment that will accept other components, such as thermal treatment packs or other element, as described in relation to FIG. 7. The geometry and properties of the sole 20 of the device 10 can allow the patient to be comfortable using the device, for example during walking. The shape of the sole 20 can provide stability or facilitate natural walking strides.

In an example, the device 10 can be fastened or fixated to the limb in a supportive manner, using a variety of fastening elements 18. Various components or panels of the device can be attached with a strap, a buckle, a ratchet, a zipper, a tie, a hook and loop fastener, a snap, a cable and cam, or other device. Fastening elements 18 can allow the patient to accurately and comfortably tighten the device onto the limb. The fastening elements 18 can allow the patient to relatively quickly remove the device 10 or replace the device 10 with substantially the same compression that was adjusted earlier.

In one example, the device is configured to be used on the forefoot while not obstructing ankle movement. For example, the device 10 can take the shape of a low cut shoe. Aspects such as fixation, gradient compression, or interchangeable thermal treatment members can be utilized, as described herein. Such a configuration can allow the patient to obtain the benefits of a device that has gradient pressure, ridged fixation, or the option for thermal therapy without having to fix the ankle or constrict the calf. This configuration can be configured to suit forefoot surgery or injury to the toes or forefoot.

The device 10 can be configured to fit various sized patients. The configuration can suit a variety of patients having different sized limbs. For example, the device 10 can be configured to have an adjustable width or length to conform to different sized feet. Such an example can utilize modular components to accommodate different anatomies.

In an example, the device 10 can be configured with a moisture wicking, thermal therapy conducive, or other comfortable fabric disposed for contact with the skin or limb. An interface material can provide comfort and perspiration control, which may facilitate device cleanliness and user comfortable.

As illustrated in FIG. 1, the device 10 can include a thermal pump 35 configured to circulate a thermal therapy fluid through thermal members, as described herein. Although illustrated on the side of the device 10, the thermal pump can be located anywhere on the device 10.

Fixation

A patient can benefit from fixation to stabilize and prevent further damage to an injured anatomy, such as a lower leg. Fixation can be achieved by a device or cast, such as device 10. Material choice, material thickness, or design geometry can help to achieve desired fixation. Fixation can include a foot neutral or ankle neutral patient foot position, wherein ankle neutral is absent of dorsal flexion, plantar flexion, inversion, or eversion. For example, an increased material thickness in boot back 12 or boot 14 at a base of the foot relative to an average boot back thickness can aid in foot neutrality. Similarly, dorsal or plantar flexion can be limited with an increase in material thickness from about the sole 20 to high-ankle height, with a marked increase in thickness near an ankle bone region. High-ankle height can, for example, include a location from the heel up to just below the knee of the patient. The sole 20 and side material thickness proportionalities can restrain ankle inversion and eversion as well.

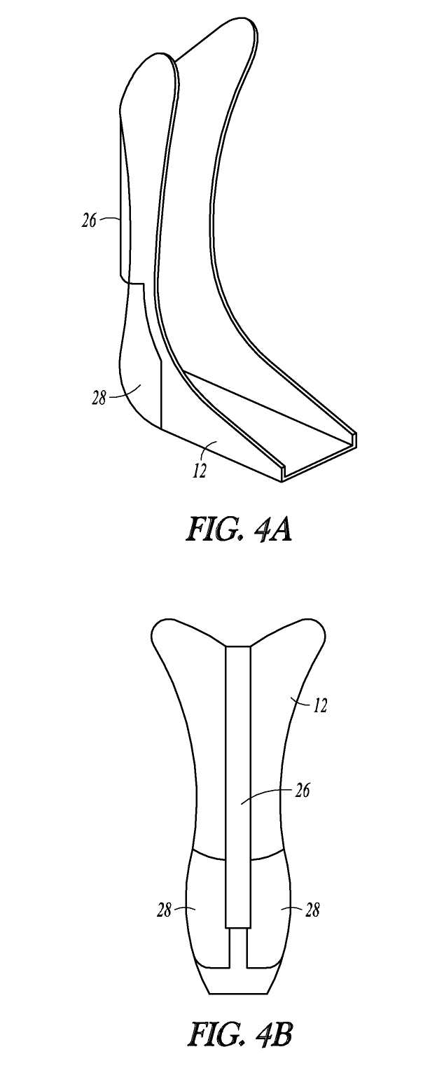

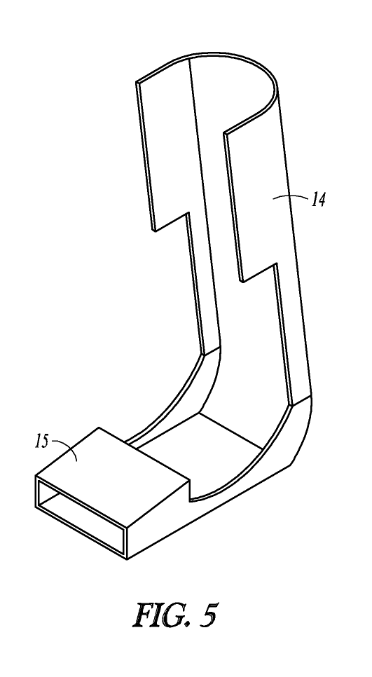

As illustrated in FIGS. 4A and 4B, the boot back 12 can include doors or compartments 28 for thermal members, as described herein. The doors or compartments 28 can be hinged, snap fit, press fit, groove and tongue, or any other means, coupled to the boot back 12. Such doors or compartments 28 can weaken the boot back 12. For structural reinforcement, a support beam 26 can be positioned along the back side of the boot back 12 to help provide support or stability. The support beam 26 can include a number of rigid materials, as described herein, including metal. The support beam 26 can be formed of a number of geometries, including but not limited to, an I-beam shape, a solid form, or a hollow configuration. For example, FIGS. 4A and 4B illustrate a solid or hollow support beam 26. As illustrated in FIG. 5, the boot 14 can include a back to aid in fixation for an ankle or high lower leg related injury. Such an example can provide structural reinforcement to the device 10 as a whole. The back of boot 14 can include a number of doors or compartments, such as door or compartments 28, to aid in thermal therapy. One example can include fixation using adjustable straps.

Compression

Compression can aid in removing excess vascular fluid from an injured region, such as the ankle or foot region. As a result of injury, the blood vessels in the affected area can expand and facilitate greater blood flow. Increased pressure applied to the ankle/foot region can decrease the diameter of the veins closest to the surface of the body so as to prevent excess amounts of vascular fluid from building up. Externally applied compression can be uniform or non-uniform in a medial to lateral direction, a proximal to distal direction, an anterior to posterior direction, or over a particular region of the affected area. In one example, the compression exhibits a gradient in magnitude of exerted force. Compression, such as a compression gradient, can facilitate blood flow around the lower foot or ankle. A compression gradient can include a spatial gradient such that compression can vary according to location within the dimension of the device on or within the affected injured area. The dimensions of the device can include the area or immediate area of the lower leg affected by the exerted compressive force. The compressive force can also provide improved contact during cooling treatment. For example, the greater the compressive force the greater the contact area between the thermal member and the skin of the patient. The static compressive force can be less than the predetermined compressive force in order to prevent capillary shut down.

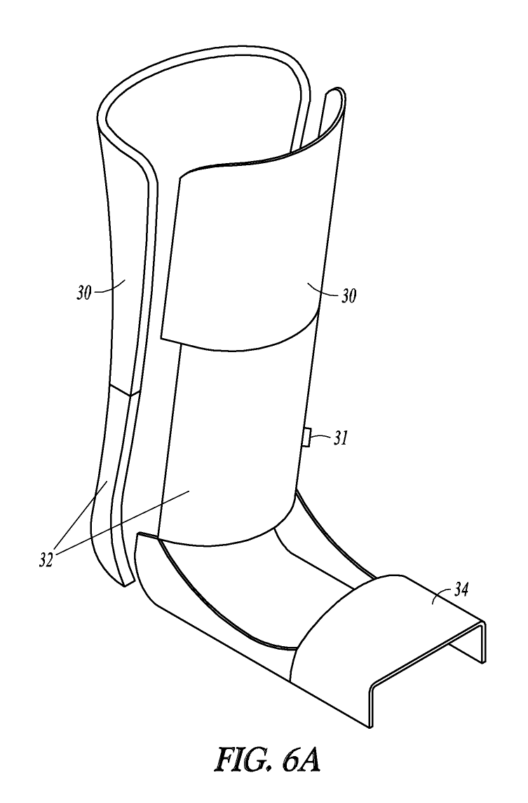

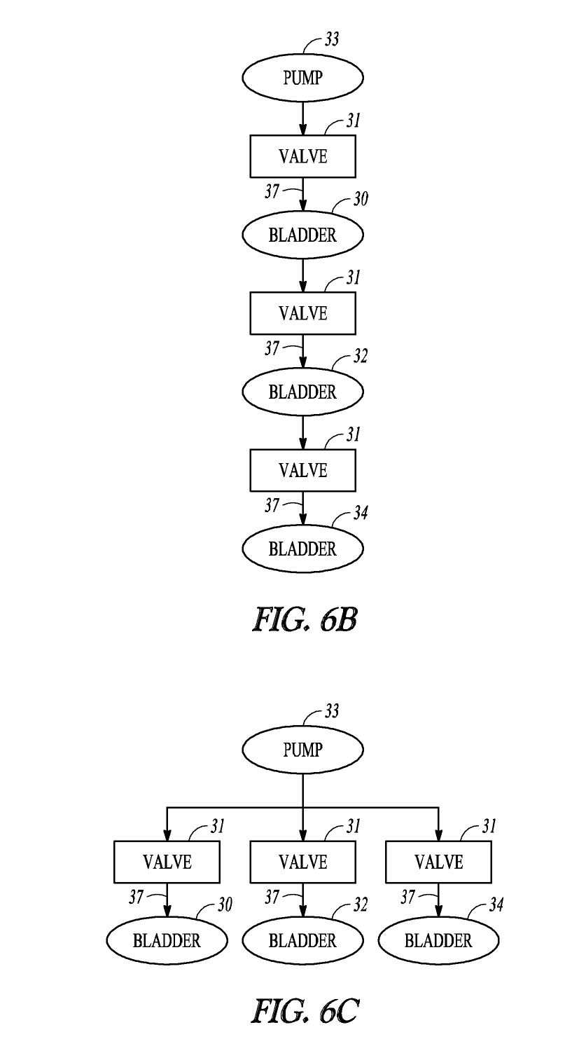

FIG. 6A illustrates an example of a device configured for a compression gradient according to the present disclosure. In an example, a compression gradient can include segmented compressive members, such as a high pressure region located on the top of the foot and a low pressure region at the upper lower leg or calf. As illustrated in FIG. 6A, the device 10 can include three compressive regions 30, 32, and 34 including at least one compressive member. A compressive member can include a bladder lining the inside of the boot back 12 or boot 14. The bladders can be configured to contain any material capable of exerting a compressive force. For example, the bladders can be formed of a flexible plastic material configured to withhold or contain a gas, such as air. The compression regions 30, 32, and 34 can each provide a unique compressive force. For example, the top compression region 30 can include a lower compressive force than the lower compression region 34. In an example, compression region 30 can be about 10 mmHg, compression region 32 can be about 15 mmHg, and compression region 34 can be about 25 mmHg.

One or more relief valves 31 can be used to achieve a selected pressure gradient. For example, each compression member or bladder within the compression regions 30, 32, and 34 or inlet lines to each compression member can include a pressure relief valve 31. A relief valve 31 can include a duckbill relief valve such that a one-way design regulates flow based on a specified cracking pressure, as is commonly understood in the art. Above the specified cracking pressure, the valve can open and allow air to flow out of the system until the internal pressure within the compression member reaches the cracking pressure. A layer of padding material can be placed between the air bladders and the patient's leg to prevent chaffing or pinching of the skin or provide comfort.

A user can inflate at least one of the bladders with a hand pump having a nozzle or needle inserted at a single inlet valve or inlet relief valve location near the middle of the device or an inlet for each of the bladders. In an example, the relief valve 31 can be the inlet relief valve. The inlet valve located on the device can attach to multiple hand pump lines. The hand pump can be attached using a quick-connect system that allows for a user-friendly interface. The quick-connect system can prevent improper connections that would result in an unintended pressure distribution, such as an increase or decrease in the predetermined compressive force exerted by the bladders. The attachment mechanism can be configured to close the bladder lines when removed after inflation, helping to prevent the air bladders from leakage during normal use. The inlet valve can also be the relief valve that helps regulate the pressure of the air bladders. Further, the relief valve can be configured to vent the bladder line to atmospheric pressure.

In an example, an integrated gradient compression pump can establish a compression gradient of decreasing pressure from the toes to the calf and prevent the user from inducing an unwanted positive pressure gradient. For example, the compressive force can be greatest at the toes and the least at the calf. Incorporating the relief valve system into a hand pump can allow for a single, easily accessible entrance location for the patient to install or remove the device. For example, the hand pump can include a mechanism to release any air over a predetermined back pressure exerted by the bladder. It can also make it easy to troubleshoot if there are any relief valve malfunctions, as opposed to embedded cascading valves in the air bladders.

A bladder can include multiple elements. The number of elements can be variable. For example, as illustrated in FIG. 6A, compression regions 30 and 32 can each include a front bladder and a rear bladder. The elements can have various shapes, such as a donut, a tube, a honeycomb, a baffle, or the like. The location of each element within the bladder can be selected based on various anatomical configurations, such as shape of the shin, thickness of the calf, angle of the ankle, or the like. The bladders can be non-removably or removably coupled to the device 10. A bladder can be positioned, shaped, or arranged in a way such that various parts of the affected area experience uniform or different amounts of pressure according to a desired gradient, in order to facilitate venous return or adequate blood flow.

A bladder can be fabricated of various materials having sufficient strength to maintain an internal pressure. Suitable materials can include a polymer, a rubber, or a plastic. The material can be selected to facilitate thermal conduction. In one example, aluminum or other thermally conductive metal or material can be used. The bladder can be configured to include a network of tubes or fluid lines incorporated throughout the device and configured for introducing thermal treatment material separate from the compression force material.

A bladder can be compliant and strong. This provides patient comfort and sufficient material strength to withstand the exerted compressive force. A thermal conductive material or tube network incorporated into the bladder or bladder material can facilitate application of thermal treatments to various anatomical structures, and help maintain good contact of the thermal therapy to the body part.

A bladder can be hollow so as to receive a filler material. The filler material can exert pressure in the bladder or exert a compressive force. An example of filler material includes a gas such as air or a liquid or solid, such as a curable elastomer, a flowable liquid, or a solid paste-like material. The filler material can include a thermal insulator or a thermal conductor. In one example, the material used to pressurize the bladder can be delivered in a controlled way to produce the desired pressure gradient. In addition, the thermal properties of the material may facilitate thermal therapy to the body part.

As illustrated in FIGS. 6B and 6C, one or more bladders 30, 32, and 34 can be interconnected by a line or tube 37 to facilitate the flow of material and application of pressure. A valve 31 can be situated on the bladder 30, 32, and 34 or in a line 37 coupled to the bladder 30, 32, and 34 to allow for various pressures at different anatomical locations. As illustrated in FIG. 6B, multiple valves 31 can be interconnected in a series configuration. As illustrated in FIG. 6C, multiple valves 31 can be interconnected in a parallel configuration. In an example, a single bladder can be inflated without adjusting pressure in the other bladders.

In an example, multiple bladders can be filled with a material using a pump 33, or other mechanism, to various pressures in a plurality of anatomical locations in order to provide a compression gradient. The pump 33 can include a piston, a bladder, a turbine, or a peristaltic mechanism. The pump 33 can include multiple chambers to deliver various materials and pressures. The pump 33 can include a valve that can be adjusted to deliver various pressures to specific bladders.

In one example, an indicator can provide a measure of pressurization levels. An indicator may be located on an outer surface of the pump 31 or may be in part or in its entirety contained in a pressure sensor for each bladder or for all bladders combined. The indicator can be manually activated or automatic and powered using a battery or other electrical power supply. The indicator can be separate from the device, incorporated within the device, or incorporated within a component of the device, such as a bladder. The pump 31 can be configured to deliver pressurized material to the bladder or can be used to deflate the bladder.

In an example, compression can be provided by straps along with foam in addition to the compression member, such as a bladder. For example, a compression gradient can be created by adjusting the tautness of individual straps at various anatomical locations. The straps are configured sufficiently wide to provide a uniform compression over the section of the leg sufficiently affected by the strap. The patient leg or foot can be measured for fit prior to using the device 10. Straps can be configured with markings that coincide with levels of compressive force. The markings can be aligned with a fastening clip, such that a patient or clinician can adjust the strap to provide the desired compressive force according to the markings. The appropriate marking can be determined by a table of pressures and lower leg/foot dimensions provided with the device.

A sensor can be used to determine when each of the bladders contains enough compression fluid such that a predetermined gradient pressure exists. An indicator light can illuminate at a predetermined pressure to, for example, indicate a particular bladder pressure. Walking can cause pressures exerted between the foot and the device to exhibit pressure spikes, which can increase the pressure within a bladder above a cracking pressure of a relief valve. Exceeding the cracking pressure of the relief valve can either deflate the air bladders once the patient started walking, or cause the gradient to vary from the designated configuration.

An example can include placement of relief valves at the hand pump. A hand pump can be configured with one pump inlet connection that incorporate three smaller individual inlets that connect to the three separate bladders. The hand pump then incorporates three separate piston chambers that each distributes air into the three air bladders. When the bladder is full, the release valve on the pump prevents air entry into the bladder. To release the air from the bladders, the pump can include an outlet valve where the patient can pump out the desired air from the device.

Thermal Therapy

Clinically appropriate thermal therapy can provide a number of benefits. Thermal therapy can include a temperature differential relative to ambient, a patient's average body temperature, or a temperature of a localized region, such as an injured appendage, of the patient. Thermal therapy can include the application of cold or hot thermal members to an affected anatomy of a patient. For example, cooling can reduce swelling, reduce associated pain, or aid in the recovery process in trauma or surgical patients. Applying heat to an injured or traumatized area can enlarge blood vessels, relax tissue, or temporarily help to relieve pain symptoms. A result of the body's natural response in an attempt to heal itself after an injury can be to increase blood flow in an affected area. The increased blood flow can translate into inflammation or swelling, which can contribute to loss of movement or decrease in function. When cold therapy is applied, tissue temperatures can be reduced and the chemical reactions within the affected area are slowed down. This in turn can cause a decrease in blood flow and reduces the risk of blood clots.

As a result of trauma or a surgical procedure, the nerve fibers in the affected area can become more active and send impulses to the spinal cord. The faster the impulses are transmitted, the higher the perception of pain can be for the patient. Cold therapy can reduce the velocity of these impulses and therefore the patient perceives lower amounts of pain. Thermal therapy can have therapeutic benefits for a patient. The thermal treatments can be applied and changed without removing the device 10 from the user, therefore maintaining stability and fixation. The various placements of the thermal treatment assure that the patient can determine a correct and comfortable place for the thermal treatment.

Cooling thermal therapy can be achieved using a removable cooling pack. For example, a cooling thermal member can be placed between a patient's lower leg and the cover 16, as illustrated in FIG. 2. The cover 16 can be removed or unhinged from the boot 14 to permit placement of a thermal member. The cover can be configured to allow access to the specified cooling zones. A thin, 0.5 mm for example, cotton insulation sleeve can be configured to fit over the thermal member to help prevent direct contact of the thermal member and the patient's skin.

During thermal therapy, an air bladder located in the cover, such as the front bladder 32 in FIG. 6, can be located on an outside of the thermal member so as to permit direct contact between the thermal member and the lower leg. Such a configuration can provide control of a thermal contact area between the thermal member and the lower leg. Due to the low thermal conductivity of air, the thermal member can be placed between the air bladders and the patient's skin. In an example, cooling thermal therapy can permit a skin temperature in a range of about 10-15° C. to be achieved at the end of a 20-minute cooling period.

Thermal therapy can be applied in a number of ways. For example, a thermal member can include a pack of material, such as a gel, a liquid, or a solid, that can be heated or cooled and then fitted into a predetermined place using a door, a compartment, or a panel in the device 10. In various examples, a chemical thermal therapy pack can uses a chemical reaction, such as an exothermic or endothermic reaction, to provide thermal therapy. The pack can be placed in a specified location using methods or structures described herein.

Thermal therapy can permit the patient to quickly or easily apply thermal treatment while stationary or while ambulatory. Thermal therapy can be constant or variable. In one example, the materials providing thermal therapy can be placed or actively pumped through a network of tubes in the device 10. For example, a device 10 can be configured to use electrically powered thermal treatment powered from a source such as a battery incorporated into the device, an external AC or DC power supply coupled by wires and an external plug, kinetic powered electric supplies, solar power, or the like. The pump can include one or more temperature sensors configured to aid in monitoring a temperature of the thermal therapy material. In various examples, thermal therapy treatments can include thermal electric cooling or resistive heating, Peltier methods, radiation, friction/vibration heating, ultrasound, or the like.

FIG. 7 illustrates a device 10 configured to provide thermal therapy using one or more thermal cavities 41, 43, and 45. Thermal cavities 41, 43, and 45 can include a hinged door, a drawer, or a panel 40, 42, and 44 in a surface of the device. In an example, a thermal member can be used to replace a door, draw, or panel 40, 42, and 44 in the device while it is being used and be replaced by a panel when the thermal component is not in use. In an example, the door, drawer, or panel, 40, 42, and 44 can be configured to secure the thermal member within a thermal cavity 41, 43, and 45.

In an example, the sole 20 of the boot back 12 can include a thermal compartment 45 to receive an inserted thermal treatment member. A high ankle thermal cavity 41 can be include in the boot back 12. A heel or ankle thermal cavity 43 can be included in the boot back 12. In an example, the thermal cavities can coincide with the compression bladders 30, 32, and 34, as illustrated in FIG. 6. For example, a thermal member can be placed in a thermal cavity such that the thermal member is between an air bladder and the skin of the patient.

An example can include cooling packs integrated within structure or geometry of the device 10. In this example, the patient removes his or her foot from the device 10 and can place ice packs in the back of the device or on top of the foot. The device 10 can then be fitted on the patient and the patient can move around. After cooling, the patient can remove the device again and remove the cooling packs.

In an example, a gel can provide thermal therapy cooling. A cooled gel can be inserted using a device similar to a pump. Once the user finishes inserting the gel, the patient can walk around. The gel can be removed from the circulating tubes, such as by suction or the like, once the thermal therapy is complete.

A thermoelectric cooler (TEC) is a device that transfers heat against the temperature gradient of two materials with the aid of electric energy. An example of the device 10 can use a TEC to aid in the cooling process. The TEC can be bonded or fused to the inside of the boot back 12 at the desired cooling regions. A thin, gel-filled bladder can be fused to the boot back and lie between the TEC and skin to cool the skin when the TEC is active. The TEC can be configured to activate by plugging an electrical cord into the device and power outlet. For safety precautions, an internal switch can deactivate the TEC after, for example, a 30-minute cooling period.

As illustrated in FIG. 8, thermal therapy can be incorporated into a modular component such as a thermal sock 50 placed in position on the user before wearing the device 10. The thermal sock 50 can include a number of pockets 52, 54, and 56 each configured to receive a thermal member. Thermal pocket 52 can include a high ankle thermal pocket, thermal pocket 54 can include a top of the foot thermal pocket, and thermal pocket 56 can include a low ankle or heel thermal pocket. In an example, the thermal sock 50 can include a thermal pocket on an arch or bottom of the foot. Each pocket can include elastic, a zipper, a button, a tie, a hook and loop fastener, or other means to secure a thermal member within the cavity. The thermal sock 50 can include a material configured to wick moisture away from the skin or insulate the lower leg. The sock 50 can provide the benefit of an easily removable, washable, or adjustable thermal therapy configuration.

General

FIG. 9 illustrates a cross sectional view of a device 10 according to the present disclosure. The device 10 can include a boot back 12, a boot 14, a cover 16, or a sole 20, as described herein. The device 10 can include padding 70 throughout, such as about the bottom of the foot or the back of the lower leg. Further padding can be included over or about a toe section or a shin section. The device 10 can include one or more thermal therapy members 76 positioned next to a lower leg of a patient. In an example, the thermal members 76 can include an outer fabric or padding to prevent direct contact with the lower leg. The thermal members 76 can provide thermal therapy as described herein. The device 10 can include one or more compression members 72, as described herein.

The compression members 72 can be positioned on a non-leg side of the thermal members 76, such that the thermal members are located between the leg and the compression members. Such a configuration can utilize the compression force exerted by the compression member 72 to aid in providing thermal therapy, as described herein. That is, the compression members 72 can be configured to adjust a thermal contact area of the thermal members 76. A thermal contact area can include an area in which thermal energy is transferred between the lower leg and the thermal member 76. For example, an increase in compressive force can provide an increase in thermal contact area between the lower leg and the thermal member, so as to increase heat transfer. If the heat transfer is too great, for example the lower leg is too cold, the patient or clinician can decrease the compressive force exerted by a compressive member 72, such as by decreasing the pressure of the compressive member 72. The compressive members 72 can be configured so as to provide a compressive spatial gradient, as described herein.

A final skin temperature, such as after a 20-minutes cooling period, can be specified to lie between 10 and 15° C. Since the final skin temperature is dependent on many variables including the applied compressive force, the maximum pressure of a bladder can be configured such that a thermal contact area permits a final skin temperature greater than 10° C. after 20 minutes. Similarly, the minimum pressure of a bladder can be configured such that the thermal contact area permits a final skin temperature less than 15° C. after 20 minutes.

One example for the compression system can include a negative spatial pressure gradient from the toes to the calf. That is, the compressive force exerted by the compressive members can decrease with increasing distance from the toes. At the toes the pressure can be configured to be 25 mmHg. In the calf region of the device, the pressure can be configured to be 10 mmHg.

A thermal therapy test can be administered to determine ranges of insulation thickness, pressure, or thermal mass that produce acceptable skin temperatures for twenty-minute cooling or heating treatments. For example, a cooling member can be applied to the right ankle and skin temperature measurements of the skin and cooling member can be recorded. Temperatures can be recorded every thirty seconds for the first two minutes and once every two minutes afterwards. During such time the foot can remain stationary, neutral positioned, and below heart level. For example, the device can be configured to achieve final skin temperature by applying a 20-mm-thick icepack, insulated by 0.5 mm of cotton, to the ankle at a pressure ranging from 10-25 mmHg for twenty minutes. However, the device can be configured in a number of different configurations and remain within the scope of this disclosure.

The non-limiting examples described herein can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device comprising:
a support structure configured to couple to a lower leg of a person and configured to at least partially restrict movement of the lower leg;
a compression member configured to exert a compressive force to a first portion of the lower leg, the compressive force having a gradient relative to a spatial position of the lower leg; and
a thermal member configured to provide thermal therapy to a second portion of the lower leg.

2. The device of claim 1 wherein the wearable support structure includes a boot back, a boot, and a cover.

3. The device of claim 2 wherein the boot back includes a support beam configured to provide stability.

4. The device of claim 2 wherein the cover includes a hinge configured to couple the cover to the boot.

5. The device of claim 2 wherein the boot back includes a hinge configured to couple a first portion of the boot back to a second portion of the boot back.

6. The device of claim 1 wherein the compression member includes at least one bladder.

7. The device of claim 6 wherein a bladder includes a push-button relief configured to vent the bladder to atmospheric pressure.

8. The device of claim 6 wherein the at least one bladder includes at least one relief valve.

9. The device of claim 8 wherein the relief valves are connected in parallel.

10. The device of claim 8 wherein the relief valves are connected in series.

11. The device of claim 1 wherein the gradient includes a low pressure region about an upper area of the lower leg.

12. The device of claim 1 wherein the gradient includes a high pressure region around a toe area of the lower leg.

13. The device of claim 1 wherein the thermal member includes a thermal contact area configurable by the compressive force.

14. The device of claim 13 wherein the compression member is configured to adjust the thermal contact area between the thermal member and the lower leg.

15. The device of claim 1 wherein the thermal member is configured to provide temperature therapy using a chemical reaction.

16. The device of claim 1 wherein the thermal member is configured to provide temperature therapy using at least one of electric cooling, resistive heating, a Peltier method, radiation, friction or vibration heating, and ultrasound.

17. A therapy method for a lower leg comprising:
stabilizing a lower leg of a person using a support structure configured to couple to the lower leg to at least partially restrict movement of the lower leg;
compressing a first portion of the lower leg using a compression member configured to exert a compressive force to at least the first portion of the lower leg, the compressive force having a gradient relative to a spatial position of the lower leg; and
thermally treating a second portion of the lower leg using a thermal member configured to provide thermal therapy.

18. The method of claim 17 further comprising using the compression member to adjust a thermal contact area between the thermal member and the lower leg.

19. The method of claim 17 further comprising circulating a thermal therapy fluid through the thermal member using a pump.

20. A device comprising:
a support structure configured to couple to a lower leg of a person to at least partially restrict movement of the lower leg, including
a boot back formed of a rigid material;
a boot configured to couple to the boot back; and
a cover configured to couple to the boot;
one or more bladders configured to exert a compressive force to the lower leg, each bladder including an inlet relief valve configured in series or parallel to provide a compression gradient relative to a spatial position on the lower leg; and
a thermal member configured to provide thermal therapy to at least a portion of the lower leg.

21. The device of claim 20 further comprising a pump configured to pressurize the one or more bladders.

22. The device of claim 20 further comprising at least one fastener configured to couple the support structure to the lower leg.
